(19) United States
(12) Patent Application Publication
(10) Pub. No.: US 2013/0143750 A1
(43) Pub. Date: Jun. 6, 2013

(54) Phoshorylation of Estrogen Receptor Alpha

(71) Applicants: Leigh C. Murphy, Winnipeg (CA); Zoann J. Nugent, Winnipeg (CA); Peter H. Watson, Victoria (CA); Georgios P. Skliris, Cambridge, MA (US)

(72) Inventors: Leigh C. Murphy, Winnipeg (CA); Zoann J. Nugent, Winnipeg (CA); Peter H. Watson, Victoria (CA); Georgios P. Skliris, Cambridge, MA (US)

(73) Assignee: The University of Manitoba, Winnipeg, MB (CA)

(21) Appl. No.: 13/501,997

(22) PCT Filed: Dec. 22, 2009
(86) PCT No.: PCT/CA2009/001887

§ 371 Date: Jul. 31, 2012

Related U.S. Application Data
(60) Provisional application No. 61/251,690, filed on Oct. 14, 2009.
(60) Provisional application No. 61/251,687, filed on Oct. 14, 2009.

(51) Int. Cl. G01N 33/74

(52) U.S. Cl. CPC G01N 33/743
USPC 506/9; 530/387.9; 506/18; 435/7.1

(57) ABSTRACT

The present invention provides use and methods markers based on the phosphorylation state of certain amino acid residues of estrogen receptor α.

### FIELD OF THE INVENTION

The present invention relates to the phosphorylation state of a number of residues on estrogen receptor α. The present invention provides methods, uses, kits, and antibodies relating to the phosphorylated estrogen receptor.

### BACKGROUND TO THE INVENTION

The hormone-dependent nature of breast cancer is well known and was first described by Beatson in 1896. Since then numerous agents have been introduced designed to either modulate estrogen receptor (ER) function or to affect the levels of circulating estrogens. Among these agents are the selective estrogen receptor modulators (e.g. tamoxifen, raloxifene, toremifene), antiestrogens (e.g. fulvestrant), luteinizing hormone-releasing hormone agonists (e.g. leuprolide, goserelin), and aromatase inhibitors (e.g. anastrozole, letrozole, exemestane).

Widespread use of endocrine therapy has led to a marked reduction in breast cancer mortality. However, a large percentage of breast cancers that are hormone receptor positive do not respond to such treatments. This may be due to intrinsic resistance or acquired resistance following prolonged use or some other, as yet unknown factor.

Endocrine therapy can cause various side-effects such as vasomotor symptoms and musculoskeletal discomfort. Occasionally the treatment can lead to more serious side effects such as thrombosis, endometrial cancer, or osteoporosis. These problems can affect the overall quality of life of the patient and can even reduce life expectancy. It is therefore important to try and avoid the unnecessary treatment with endocrine therapy and to allow the early adoption of alternative treatment strategies for patients with endocrine-resistant tumours.

Attempts have been made to predict responsiveness of ER+ breast cancers to endocrine therapy. See, for example, U.S. Pat. No. 7,105,642 which describes a monoclonal antibody specific for ER α having a phosphorylated serine residue at the 118 position. The presence of phosphorylation at Ser118 is said to have predictive value as to the progression and outcome of the disease or the response of the disease to targeted therapy. Phosphorylation at Ser118 improves the chances of survival in ER+ breast cancer (Yamashita H, Nishio M, Toyama T, et al: Low phosphorylation of estrogen receptor α (ER α) serine 118 and high phosphorylation of ER α serine 167 improve survival in ER-positive breast cancer. Endocr Relat Cancer 15:755-63, 2008; Jiang J, Sarwar N, Peston D, et al: Phosphorylation of estrogen receptor-alpha at Ser167 is indicative of longer disease-free and overall survival in breast cancer patients. Clin Cancer Res 13:5769-5776, 2007).

### SUMMARY OF THE INVENTION

The present invention provides a method of detecting the presence of phosphorylation at certain residues of ER α.

The invention further provides the use of the detection of phosphorylation at certain residues of ER α for predicting response to endocrine therapy.

The invention further provides a method of predicting treatment outcomes for breast cancers treated with endocrine therapy.

The invention further provides a method of diagnosis and a method for optimising treatment.

The invention further provides computer programs for implementing the present method of diagnosis as well as computers running such programs.

The invention further provides kits comprising antibodies for detecting phosphorylation at certain residues of ER α.

All references cited herein are hereby incorporated herein by reference.

### BRIEF DESCRIPTION OF THE DRAWINGS

FIG. 1 A shows Kaplan-Meier estimates of overall survival from breast cancer specific death with respect to expression of PR (LBA, PR high >20 fmol/mg protein(top left); p-S282-ERα (high >25% H-score, top right); p-T311-ERα (high >25% H-score, bottom left); p-S118-ERα (positive>0 H-score, bottom right).

FIG. 1B shows Kaplan-Meier estimates of relapse free survival from breast cancer recurrence or breast cancer specific death with respect to expression of PR (LBA, PR+ve>20 fmol/mg protein(top left); p-S282-ERα (top right); p-T311-ERα (bottom left); p-S118-ERα (bottom right).

P value represents the significance of the hazard ratio for each factor.

FIG. 2A shows Kaplan-Meier estimates of overall survival from breast cancer specific death with respect to phosphorylation score=P⁷ score (high≧3). 2B Kaplan-Meier estimates of relapse free survival from breast cancer recurrence or breast cancer specific death with respect to P⁷ score (high≧3).

P values represent the significance of the hazard ratio for each factor.

### DETAILED DESCRIPTION OF THE INVENTION

The present invention is based on investigations to determine the relationship of the phosphorylation state of various sites of estrogen receptor (ER α) to clinical outcome and response to endocrine therapy, such as tamoxifen, in human breast cancer.

In accordance with the present invention there are provided antibodies that bind to ER α only when it is phosphorylated at certain amino acid residues. Phosphorylation at these sites is of particular interest as they have predictive value when assessing treatment outcomes, tumour progression, and/or responsiveness to endocrine therapy.

Detection of the phosphorylation may provide valuable information about the mechanisms of resistance to ER-inhibitors, such as tamoxifen.

Also provided are methods of using antibodies to detect ER α phosphorylation and activation in a biological sample or test tissue suspected of containing phosphorylated ER α or having altered ER α activity, as further described below.

The phosphorylation state of the following target residues was assessed—Serine 104/106; Serine 118; Serine 167; Serine 282; Serine 294; Threonine 311; and Serine 559.

The antibodies used for the assessment were as follows: p-S104/106-ERα, p-S282-ERα, p-S294-ERα, p-T311-ERα and p-S559-ERα were rabbit polyclonal affinity purified antibodies (1 mg/ml, provided by Bethyl Laboratories, Montgomery, Tex., USA); p-S118-ERα (16J4, Cell Signaling, USA); p-S167-ERα (Abcam, Cambridge, Mass., USA) and ERα (NCL-ER, clone 6F11, Novocastra Laboratories, Newcastle, UK).

The invention provides ER α phosphospecific antibodies that bind when ER α is phosphorylated at the target residues such that the phosphorylated state may be distinguished from the unphosphorylated state. For example, it may be that the antibodies do not substantially bind to ER α when not phosphorylated at the target residues.

The term “antibody” or “antibodies” as used herein refers to all types of immunoglobulin's, including IgG, IgM, IgA, IgD, and IgE. The antibody may be of any species of origin, including (for example) mouse, rat, rabbit, horse, or human, or may be chimeric antibodies. It is preferred that the antibodies be a monoclonal.

The term “ER α antibody” means an antibody that binds phosphorylated ER α as disclosed herein. The term “does not bind” with respect to such an antibody means does not substantially react with as compared to binding to phospho-ER α.

The present invention provides methods of assessing a biological sample for the phosphorylation state of the target residues of ER α. The methods disclosed herein may be employed with any suitable biological sample. For example, biological samples taken from human subjects for use in the methods herein are generally serum, blood plasma, fine needle aspirant, ductal lavage, bone marrow sample, ascites fluid, tissue samples (e.g., a biopsy tissue), such as skin or hair follicle or tumour tissue.

The present invention provides a method for detecting phosphorylated ER α in a biological sample by (a) contacting a biological sample suspected of containing ER α phosphorylated at one or more of the target residues with phospho-ER α antibody or antibodies under conditions suitable for formation of an antibody-ER α complex, and (b) determining the presence or absence of said complex. Biological samples may be obtained from subjects suspected of having a disease involving altered ER α expression or activity (e.g., breast cancer). Samples may be analyzed to monitor subjects who have been previously diagnosed as having a disease involving altered ER α expression or activity (e.g., breast cancer), to screen subjects who have not been previously diagnosed, or to monitor the desirability or efficacy of therapeutics targeted at ER α (e.g. tamoxifen).

The present invention provides a method for profiling ER α activation in a test tissue suspected of involving altered ER α activity, by (a) contacting the test tissue with phospho-ER α antibody or antibodies under conditions suitable for formation of an antibody-ER α complex, (b) determining the presence of said complex in the sample, and (c) comparing the presence of phosphorylated ER α detected in step (b) with the presence of phosphorylated ER α in a control tissue.

The methods described above are applicable to examining tissues or samples from cancers characterized by ER α activity, such as breast cancers, in which phosphorylation of ER α at the target residues has predictive value as to the progression and/or outcome of the disease and/or the response of the disease to certain therapy. It is anticipated that the present methods will have diagnostic utility in diseases characterized by, or involving, altered ER α phosphorylation. The methods are applicable, for example, where samples are taken from a subject that has not been previously diagnosed as having characterized by, or involving, altered ER α phosphorylation (e.g. breast cancer) and the methods are employed to help diagnose the disease. Additionally, the methods are applicable where a subject has been diagnosed with a disease characterized by, or involving, altered ER α phosphorylation (e.g. breast cancer) but has not yet undergone treatment and the methods may be employed in aiding in the selection of an appropriate therapy. The methods are applicable where a subject has been diagnosed with a disease characterized by, or involving, altered ER α phosphorylation (e.g. breast cancer) and the methods are employed to monitor the progression of the disease. The methods may be employed to assess risk of the subject developing a disease characterized by, or involving, altered ER α phosphorylation (e.g. breast cancer). Such an assay may be employed to identify subjects who would be most likely to respond to therapeutics targeted at inhibiting ER α activity.

The present invention provides a method for identifying a compound which modulates phosphorylation of ER α, by (a) contacting the test tissue with the compound, (b) contacting the test tissue with phospho-ER α antibody or antibodies under conditions suitable for formation of an antibody-ER α complex and determining the level of phosphorylated ER α in said test tissue, and (c) comparing the level of phosphorylated ER α detected in step (b) with the presence of phosphorylated ER α in a control tissue not contacted with the compound.

Assays carried out in accordance with methods herein may be homogeneous assays or heterogeneous assays. In a homogeneous assay the immunological reaction usually involves an ER α antibody, a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay, the reagents are usually the sample of interest, an ER α antibody, and suitable means for producing a detectable signal. The antibody is generally immobilized on a support, such as a bead, plate or slide, and contacted with the sample suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. p-ER α antibodies disclosed herein may be conjugated to a solid support suitable for a diagnostic assay (e.g., beads, plates, slides or wells formed from materials such as latex or polystyrene) in accordance with known techniques, such as precipitation. Antibodies of the invention, or other ER α binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

p-ER α antibodies disclosed herein may be used in a flow cytometry assay to determine the activation status of ER α in patients before, during, and after treatment with a drug targeted at inhibiting ER α phosphorylation at one or more of the target residues. For example, fine needle aspirants from ductal lavages or dispersed solid tumour biopsies from patients may be analyzed by flow cytometry for ER α phosphorylation, as well as for markers identifying various epithelial cell types.

Diagnostic kits for carrying out the methods disclosed above are also provided by the invention. Such kits comprise at least one p-ER α monoclonal antibody. For example, the kits may comprise antibodies to p-S282-ERα; p-S294-ERα; p-T311-ERα; p-S559-ERα; p-S104/106-ERα; or combinations thereof. The present kits may also comprise antibodies to p-S118-ERα and/or p-S167-ERα.

Preferred kits comprise four or more of the phosphor-ER α antibodies. More preferably five or more. Even more preferably at least six.

The antibodies may be coupled to a solid support. The kits may comprise ancillary agents such as buffering agents and protein stabilizing agents, e.g., polysaccharides and the like. Diagnostic kits may further include, where necessary, other members of the signal-producing system of which system the detectable group is a member (e.g., enzyme substrates), agents for reducing background interference in a test, control reagents, apparatus for conducting a test, and the like. The present kits may be packaged in any suitable manner, typically with all elements in a single container along with a sheet of printed instructions for carrying out the test.

The present invention encompasses modifications and variations of the methods taught herein which would be obvious to one of ordinary skill in the art. Unless otherwise specified, all references referred to herein are incorporated into this specification. The following examples are provided to further illustrate the invention.

### EXAMPLES

Tissue Microarrays (TMAs)

All primary invasive breast cancers used in the present study were obtained from the Manitoba Breast Tumor Bank (MBTB, CancerCare Manitoba and University of Manitoba). Samples were selected using criteria of ER positive (ligand binding assays (LBA) >3 fmol/mg protein) and treatment with surgery with or without radiation and then tamoxifen therapy cases were re-reviewed on hematoxylin and eosin (H&E) sections by pathologists to confirm block composition and select areas for TMA coring. Estrogen receptor (ER+) and progesterone receptor (PR) positive status was defined by ligand binding assay (LBA) (scores of >3 fmol/mg protein and >20 fmol/mg protein, respectively). Four hundred and fifty cases were represented on the original TMAs however due to exhaustion of tumour cores from previous use of the TMAs, or incomplete data for some cases, the number (n) of tumors analyzed for some of the markers was less than 450.

Antibodies

The antibodies used for immunohistochemistry (IHC) have been validated previously^{5,6} and were as follows: p-S104/106-ERα, p-S282-ERα, p-S294-ERα, p-T311-ERα and p-S559-ERα were rabbit polyclonal affinity purified antibodies (1 mg/ml, provided by Bethyl Laboratories, Montgomery, Tex., USA); p-S118-ERα (16J4, Cell Signaling, USA); p-S167-ERα (Abcam, Cambridge, Mass., USA) and ERα (NCL-ER, clone 6F11, Novocastra Laboratories, Newcastle, UK) antibodies were used as previously described^{3,8}.

Immunohistochemistry (IHC) for TMAs was performed as described previously⁸. Serial sections (5 μm) of the TMAs were stained with anti-ERα, anti-p-S104/106-ERα, anti-p-S118-ERα, anti-p-S167-ERα, anti-p-S282-ERα, anti-p-S294-ERα, anti-p-T311-ERα and anti-p-S559-ERα antibodies as previously described⁶. Briefly, sections were submitted to heat-induced antigen retrieval in the presence of a citrate buffer (CC1, Ventana Medical Systems, Ariz., USA) using an automated tissue immunostainer (Discovery Staining Module, Ventana Medical Systems, Ariz., USA).

Slides were viewed and scored using standard light microscopy. IHC-scores derive from a semi-quantitative assessment of both staining intensity (scale 0-3) and the percentage of positive cells (0-100%). These two scores when multiplied generate an overall IHC score of 0-300. Only nuclear staining was evaluated and scored as positive nuclear immuno-staining for ERα, p-S104/106-ERα, p-S118-ERα, p-S167-ERα, p-S282-ERα, p-S294-ERα, p-T311-ERα and p-S559-ERα protein expression. TMAs were evaluated independently by up to three investigators and where divergence was found, cases were re-evaluated to reach consensus. Since no relevant clinical cut-off points are presently reported for any of the phosphorylated ERα sites in the literature, positive results reported in this study were solely based on IHC-scores equivalent to the 25% percentile⁶. Relapse Free Survival (RFS) was defined as time to first recurrence or death due to breast cancer (censors were other death) and overall survival (OS) was defined as time to death due to breast cancer (censors were other death).

Statistical Methodology

Survival analysis was undertaken using Cox regression analyses to examine hazard ratios. Each model was tested and all complied with the assumption of proportional hazard. Statistical analyses were performed using SAS™version 9.1.

Results

The clinical-pathological characteristics of the study cohort are shown in Table 1. Primary tumours were only considered in this cohort when they were positive for ER using both ligand binding assay (LBA >3 fmol/mg protein) and IHC. All cases had been treated with surgery with or without radiation followed by tamoxifen therapy. The median follow-up period was 99 months (range 9 to 217 months).

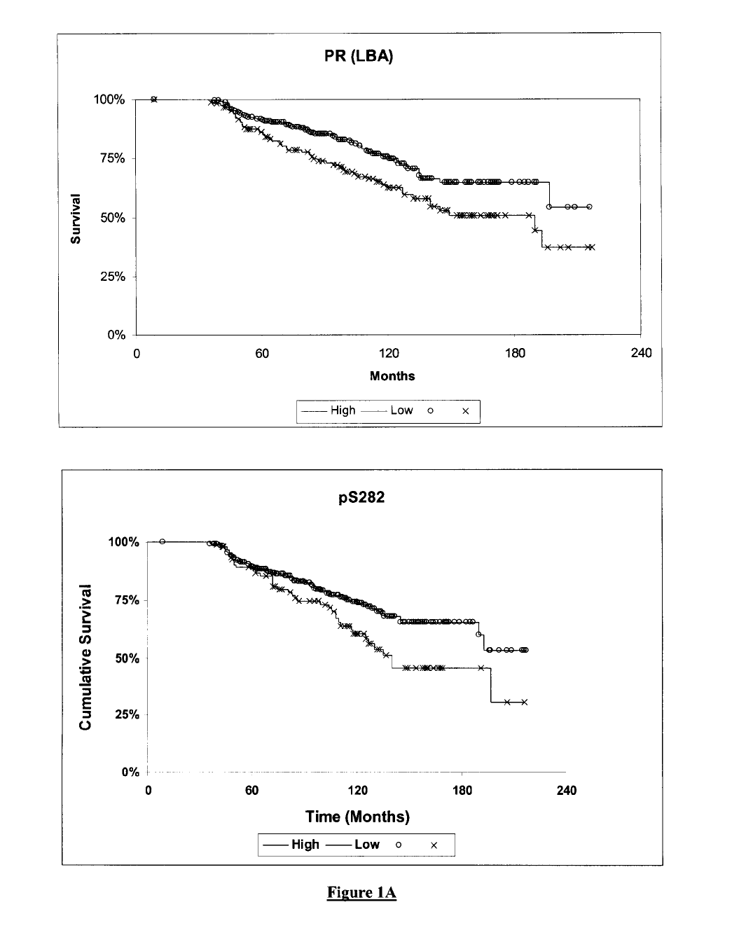

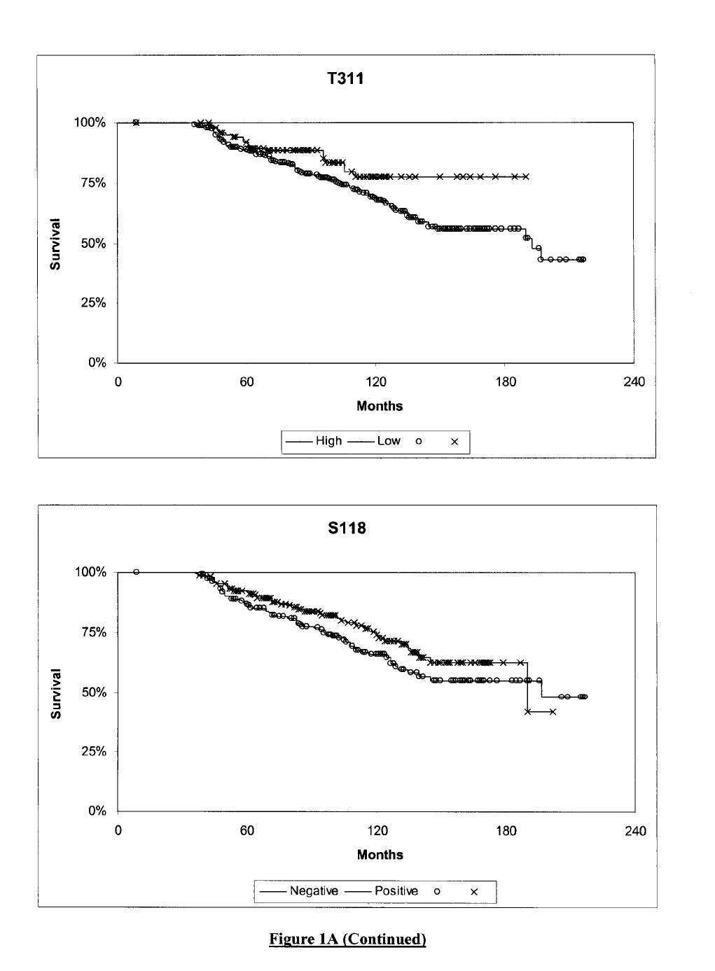

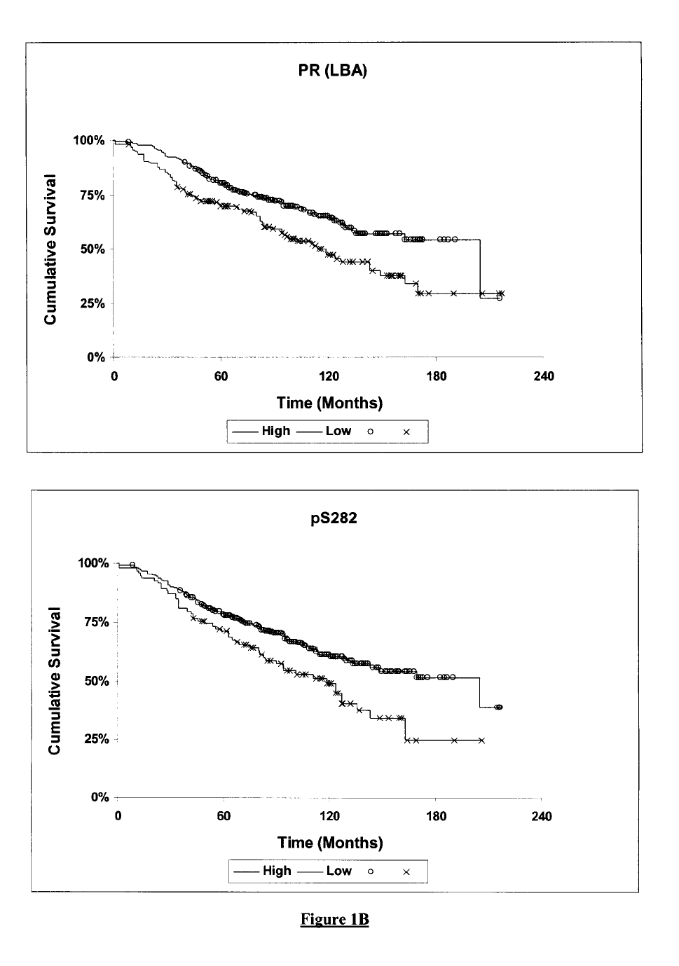

Single predictor or univariate analysis of this cohort is shown in Table 2 and Table 3 for overall survival (OS) from death due to breast cancer and recurrence free survival (RFS), respectively. Large tumour size, node positivity, high grade and PR negative status (FIGS. 1A & B) were all significantly associated with reduced OS from breast cancer death and reduced RFS. These are previously identified predictors of clinical outcome and provide evidence that our study cohort is comparable to others published in the literature despite a bias for tumour size due to the nature of the MBTB collection^{7,9} and having been selected for ER+status.

In addition the current data confirm our earlier findings using a smaller cohort^{2,3}, that detection of p-S118-ERα is associated with a significantly longer RFS (HR=0.693, P=0.0283, n=370) but not with OS in patients treated with tamoxifen (HR=0.742, P=0.135, n=370) (FIGS. 1A & B).

High levels of phosphorylation at site p-S282-ERα(>25% H-score) are significantly associated with both a longer RFS (HR=0.613, P=0.0039, n=409) (FIG. 1B) as well as OS (HR=0.615, P=0.0148, n=409) (FIG. 1A) on tamoxifen from death due to breast cancer.

Higher levels of phosphorylation at T311-ERα (>25% H-score) are significantly associated with a shorter RFS (HR 1.572, P=0.0302 n=409) (FIG. 1 B) and a borderline significant shorter OS (HR=1.674, P=0.0512, n=409) (FIG. 1A) on tamoxifen from death due to breast cancer. This phosphorylation site is, therefore, associated with a poor clinical outcome to tamoxifen.

Although not statistically significant, the HR for higher levels of p-S559-ERα for OS was 1.231 and for RFS was 1.107. This outcome pattern resembles that for p-T311-ERα

These results together support the presence of multiple phosphorylated forms of ERα in any one ER+ breast tumour biopsy, suggested to us that a phosphorylation code for ERα may exist. By analogy to the “histone code” that provides a measure of the functionality of the protein over and above measurement of the total protein itself and since multiple pathways can impact directly or indirectly on ERα to affect activity (e.g. ligand dependent versus independent; agonist versus antagonist activity of SERMs such as tamoxifen etc) it is possible that a measure of the balance between good and bad phospho-epitopes on ERα might provide a more precise predictor of outcome to tamoxifen and possibly other endocrine therapies.

To address this hypothesis a phospho-epitope ERα score was developed that would reflect the functional balance with respect to clinical outcome of all the sites measured. Scores were dichotomized using the 25^{th} percentile H-score for the epitope being considered as a cutpoint to categorize each into positive (>25^{th} percentile value of 1) or negative status. It should be noted that in some cases (e.g. p-S104/6-ERα, p-S118-ERα, p-S167-ERα, p-S294-ERα) the actual 25% H-score was 0. Since in our analysis above, p-S104/6-ERα, p-S118-ERα, p-S167-ERα, p-282-ERα and p-S294-ERα are associated with a HR below 1, they were considered “good” factors and their detection given a negative 1 (-1) value. Since p-T311-ERα and p-S559-ERα were associated with an HR above 1, they are considered “bad” factors and their detection was given a positive 1 (+1) value.

The sum of individual scores and a constant value (v=number of epitopes tested−2) was calculated to determine the P⁷-ERα score with the expectation that low P⁷-ERα scores would indicate better outcomes. For example, a tumour positive for pS104/6-ERα, p-S118-ERα, p-S167-ERα, p-S282-ERα, and p-S294-ERα and positive for p-T311-ERα and p-S559-ERα would receive a P⁷-ERα score of 5−1−1−1−1−1+1+1=2; a tumour positive for p-S118-ERα and p-T311-ERα and negative for all the other sites would receive a score=5−1+1=5; and a tumour positive for pS104/6-ERα, p-S118-ERα, p-S167-ERα, p-S282-ERα and p-S294-ERα would receive a score=5−1−1−1−1−1=0.

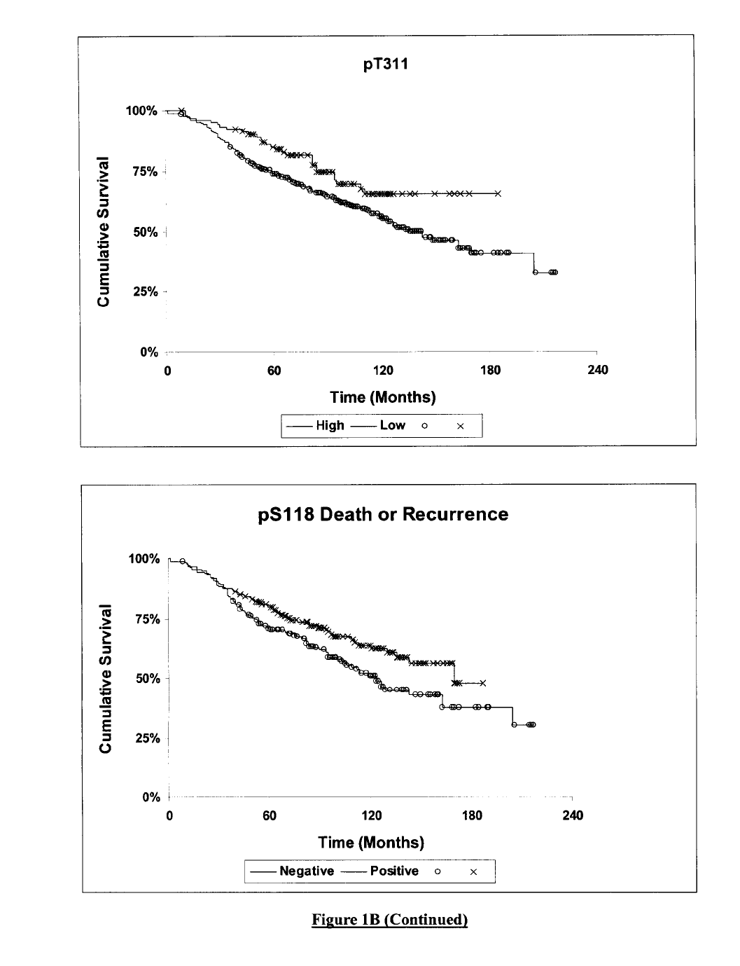

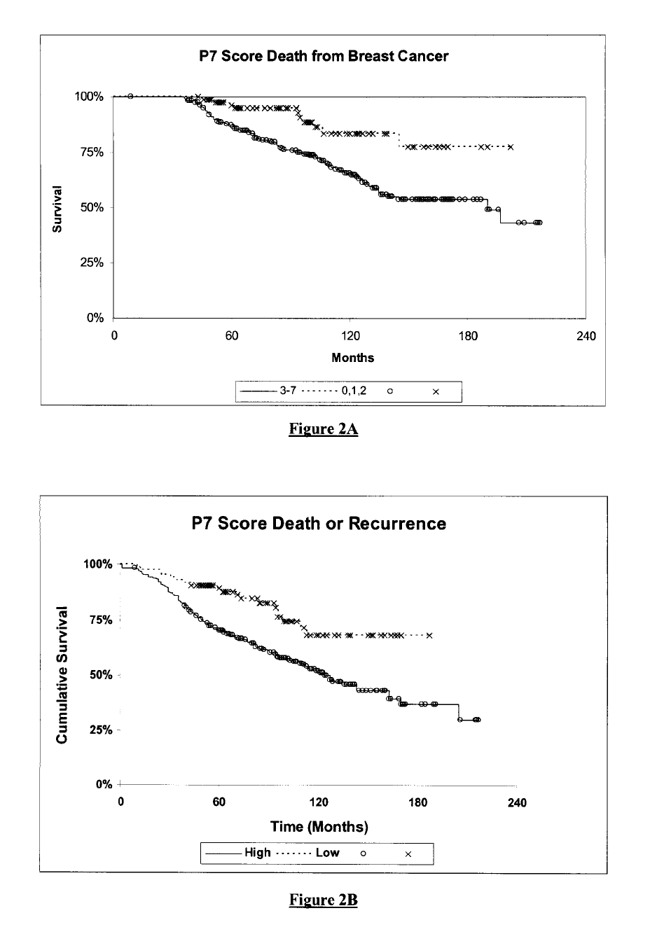

Using this approach we found that tumours with phosphorylation scores of 3 or greater identified a population of patients who had a significantly worse outcome on tamoxifen than those whose scores were below 3 (FIG. 2A for OS and FIG. 2B for RFS). In univariate/single predictor analysis the HR was 2.782 for OS (P=0.0022, n=340) (Table 2), and for RFS the HR was 2.225 (P=0.0012, n=340) (Table 3).

In order to determine if predictors identified in the univariate analysis were independent predictors, a multi-predictor (multivariate) analysis with backward selection was undertaken. The best predictors for OS from death due to breast cancer as shown in Table 4 are size, node status and phosphorylation score. The HR for the P⁷-ERα score was 2.235 (P=0.0175, n=335) for OS from death due to breast cancer on tamoxifen. For RFS (Table 5) the best predictors are size, node status, grade, PR status and P⁷-ERα score.

The analysis was also performed using a single predictor analysis using only cases in which all the variables included in the analysis were available for every case (all in data) thus reducing case numbers to 254. The results are presented in Tables 6 and 7 for OS and RFS, respectively. Size and P⁷-ERα score remain significant, with the HR for P⁷-ERα score being 2.979 for OS from death due to breast cancer (P=0.0293, n=254). For RFS (Table 7) size, grade, PR status and P⁷-ERα score are significant, with the HR for P⁷-ERα score being 2.283 (P=0.0298, n=254). A multivariate analysis with backward selection in this group identified the best predictors as size, node status and P⁷-ERα score as significant (Table 8) for OS, and size, node status, grade and PR status as significant for RFS (Table 9).

Modulation of phosphorylation of nuclear receptors, including steroid hormone receptors is known to significantly affect receptor function^{l}” and importantly has been suggested to affect the responsiveness of steroid receptors such as ER to ligands which are selective estrogen receptor modulators e.g. tamoxifen^{11,12}. Alteration of cell-signalling pathways occurs during breast tumorigenesis and breast cancer progression and involves significant modulation of kinase and phosphatase activities. This knowledge has led to the suggestion that changes in cell signaling that lead to altered phosphorylation of ER may underlie in part the development of altered sensitivity to estrogenic ligands and/or the development of resistance to endocrine therapies.

Three significant novel observations have been made. Firstly, high levels of the novel p-S282-ERα in univariate analysis significantly predicted for a better outcome, both RFS and OS, in patients treated with tamoxifen therapy. Secondly, high levels of p-T311-ERα significantly predicted a poor outcome for patients treated with tamoxifen. Thirdly, closer examination of the hazard ratios for individual phosphorylation sites measured in this study suggested that there were two groups of p-epitopes: one which was associated with a better outcome on tamoxifen and the other which was associated with a poor outcome on tamoxifen. There may be a phosphorylation code for ERα that more precisely reflects the functional status of ERα regulated events in tumours and potentially more precisely predicts for treatment response. A phosphorylation score was developed that incorporated all phosphorylation sites with ‘poor prognosis’ sites increasing the score and ‘good prognosis’ sites reducing the score. Our results suggest that a low phosphorylation score (reflecting the balance of good sites over bad sites) is a significant independent predictor of better overall survival in patients on tamoxifen.

The ability to detect specific nuclear staining of all these phospho-specific sites in some ER+ breast tumor biopsy samples provides strong support for their relevance in vivo and therefore a strong rationale to study their roles in ERα action. However, little is known about the role of S282 in ERα activity. Williams et al.,⁴ identified S282-ERα as a novel site phosphorylated after E2 stimulation in Cos transfected cells as well as in human breast cancer cell lines, endogenously expressing ERα. S282 is located within the hinge region of ERα that is thought to encode an important nuclear localization signal (256-303) as well as being at the start of a region (282-351) containing an autonomous transcriptional activation activity (AF2a) identified by Norris et. al., in yeast and some mammalian cells¹³. Little is known of the function of phosphorylation at this site except it can modestly affect estrogen regulated transcriptional activity⁴, is located in a CK2 phosphorylation motif and can be phosphorylated by CK2 in vitro⁴.

The presence of multiple phosphorylation sites on ERα that may have differential effects on activity, raises the possibility that phospho-profiling of ERα or an “ERα phospho-code” similar to a histone code may exist and provide a more precise prediction of treatment response to endocrine therapies.

TABLE 1
Clinical-Pathological Characteristics of the Study Cohort

| Factor | No. | % |
|---|---|---|
| PR >20 fmol/mg protein | | |
| +ve | 261 | 62 |
| −ve | 160 | 38 |
| Grade | | |
| Low | 118 | 28 |
| Inter | 260 | 62 |
| High | 42 | 10 |
| Size | | |
| ≦2.5 cm | 237 | 56 |
| >2.5 cm | 185 | 44 |
| Age | | |
| ≦50 | 31 | 7 |
| >50 | 389 | 93 |
| Node | | |
| − | 219 | 53 |
| + | 196 | 47 |

TABLE 2
Univariate Analysis of Factors Associated with Overall Survival
(Death due to breast cancer)

| Predictor | N | HR | Lower | Upper | P |
|---|---|---|---|---|---|
| Age ≧50 years | 420 | 1.673 | 0.735 | 3.806 | 0.22 |
| Size >2.5 cm | 422 | 2.272 | 1.564 | 3.299 | <0.0001* |
| Node + | 415 | 2.063 | 1.418 | 3.003 | 0.0002* |
| Grade | 420 | 1.384 | 1.024 | 1.87 | 0.0347* |
| PR (LBA) >20 fmol/mg | 421 | 0.606 | 0.421 | 0.871 | 0.0068* |
| pS104/6+ | 300 | 0.869 | 0.566 | 1.333 | 0.5194 |
| pS118 + | 370 | 0.742 | 0.501 | 1.097 | 0.1349 |
| pS167+ | 399 | 0.945 | 0.646 | 1.381 | 0.7685 |
| pS282+ | 409 | 0.615 | 0.417 | 0.909 | 0.0148* |
| pS294 + | 409 | 0.951 | 0.654 | 1.386 | 0.795 |
| pT311+ | 409 | 1.674 | 0.997 | 2.809 | 0.0512 |
| pS559+ | 410 | 1.231 | 0.804 | 1.887 | 0.339 |
| Phospho⁷- | 340 | 2.782 | 1.446 | 5.352 | 0.0022* |
| score 3+ | | | | | |

HR = hazard ratio

TABLE 3
Univariate Analysis of Factors associated with Recurrence Free Survival
(RFS)

| Predictor | N | HR | Lower | Upper | P |
|---|---|---|---|---|---|
| Age ≧50 years | 420 | 1.565 | 0.799 | 3.067 | 0.1917 |
| Size >2.5 cm | 422 | 1.85 | 1.361 | 2.514 | <0.0001* |
| Node + | 415 | 2.018 | 1.476 | 2.758 | <0.0001* |
| Grade | 420 | 1.448 | 1.115 | 1.880 | 0.0055* |
| PR (LBA) >20 fmol/mg | 421 | 0.599 | 0.442 | 0.813 | 0.0010* |
| pS104/6+ | 300 | 0.905 | 0.630 | 1.299 | 0.5889 |
| pS118 + | 370 | 0.693 | 0.499 | 0.962 | 0.0283* |
| pS167+ | 399 | 0.907 | 0.659 | 1.247 | 0.5471 |
| pS282+ | 409 | 0.613 | 0.440 | 0.855 | 0.0039* |
| pS294 + | 409 | 0.860 | 0.628 | 1.177 | 0.3454 |
| pT311+ | 409 | 1.572 | 1.044 | 2.366 | 0.0302* |
| pS559+ | 410 | 1.107 | 0.783 | 1.565 | 0.5664 |
| Phospho⁷- | 340 | 2.225 | 1.371 | 3.610 | 0.0012* |
| score 3+ | | | | | |

TABLE 4
Multivariate Analysis (Best Predictor Model with Backward Selection of
Factors Associated with Overall Survival (death due to breast cancer)

| Predictor | N | HR | Lower | Upper | P |
|---|---|---|---|---|---|
| Size >2.5 cm | 335 | 1.971 | 1.307 | 2.973 | 0.0012* |
| Node + | 335 | 1.66 | 1.092 | 2.523 | 0.0176* |
| Phospho⁷- | 335 | 2.235 | 1.151 | 4.338 | 0.0175* |
| score 3+ | | | | | |

TABLE 5
Multivariate Analysis (Best Predictor Model with Backward Selection) of
Factors Associated with RFS.

| Predictor | N | HR | Lower | Upper | P |
|---|---|---|---|---|---|
| Size >2.5 cm | 332 | 1.744 | 1.238 | 2.457 | 0.0015* |
| Node + | 332 | 1.629 | 1.147 | 2.315 | 0.0065* |
| Grade | 332 | 1.362 | 1.030 | 1.801 | 0.0301* |
| PR (LBA | 332 | 0.634 | 0.452 | 0.889 | 0.0083* |
| >20 fmol/mg) | | | | | |
| Phospho⁷- | 332 | 1.713 | 1.027 | 2.859 | 0.0392* |
| score 3+ | | | | | |

TABLE 6
Univariate Analysis of Factors Associated with Overall Survival (death
due to breast cancer) using only cases where all variables were
available for each case.

| Predictor | N | HR | Lower | Upper | P |
|---|---|---|---|---|---|
| Age ≧50 | 254 | 1.19 | 0.417 | 3.4 | 0.7453 |
| years | | | | | |
| Size >2.5 cm | 254 | 2.123 | 1.336 | 3.373 | 0.0014* |
| Node + | 254 | 1.552 | 0.942 | 2.556 | 0.0847 |
| Grade | 254 | 1.333 | 0.936 | 1.898 | 0.1106 |
| PR (LBA) >20 fmol/mg | 254 | 0.683 | 0.432 | 1.081 | 0.1036 |
| pS104/6+ | 254 | 1.109 | 0.646 | 1.905 | 0.7068 |
| pS118 + | 254 | 0.952 | 0.571 | 1.587 | 0.8514 |
| pS167+ | 254 | 1.176 | 0.721 | 1.916 | 0.5162 |
| pS282+ | 254 | 0.799 | 0.485 | 1.317 | 0.3787 |
| pS294 + | 254 | 1.260 | 0.761 | 2.087 | 0.3693 |
| pT311+ | 254 | 0.666 | 0.333 | 1.331 | 0.2498 |
| pS559+ | 254 | 1.609 | 0.866 | 2.989 | 0.1324 |
| Phospho- | 254 | 2.979 | 1.116 | 7.952 | 0.0293* |
| score 3+ | | | | | |

TABLE 7
Univariate Analysis of Factors Associated with RFS were all variables
were available for each case

| Predictor | N | HR | Lower | Upper | P |
|---|---|---|---|---|---|
| Age ≧50 | 254 | 1.069 | 0.459 | 2.486 | 0.8777 |
| years | | | | | |
| Size >2.5 cm | 254 | 1.791 | 1.222 | 2.625 | 0.0028* |
| Node + | 254 | 1.364 | 0.892 | 2.085 | 0.152 |
| Grade | 254 | 1.411 | 1.037 | 1.922 | 0.0286* |
| PR (LBA) >20 fmol/mg | 254 | 0.590 | 0.403 | 0.864 | 0.0067* |
| pS104/6+ | 254 | 1.236 | 0.776 | 1.968 | 0.3719 |
| pS118 + | 254 | 1.004 | 0.647 | 1.560 | 0.9847 |
| pS167+ | 254 | 1.211 | 0.798 | 1.836 | 0.3688 |
| pS282+ | 254 | 0.78 | 0.509 | 1.198 | 0.2566 |
| pS294 + | 254 | 1.298 | 0.840 | 2.005 | 0.2394 |
| pT311+ | 254 | 0.818 | 0.460 | 1.454 | 0.4928 |
| pS559+ | 254 | 1.321 | 0.804 | 2.171 | 0.2716 |
| Phospho⁷- | 254 | 2.283 | 1.084 | 4.807 | 0.0298* |
| score 3+ | | | | | |

TABLE 8
Multivariate Analysis (Best Predictor Model) with Backward Selection of
Factors Associated with Overall Survival (death due to breast cancer)
using only those cases where each variable was available for each case.

| Predictor | N | HR | Lower | Upper | P |
|---|---|---|---|---|---|
| Size >2.5 cm | 254 | 2.017 | 1.293 | 3.147 | 0.002* |
| Node + | 254 | 1.572 | 1.002 | 2.467 | 0.0488* |
| Phospho⁷- | 254 | 2.775 | 1.207 | 6.382 | 0.0163* |
| score 3+ | | | | | |

TABLE 9
Multivariate Analysis (Best Predictor Model) with Backward Selection of
Factors Associated with RFS using only those cases where each variable
was available for each case.

| Predictor | N | HR | Lower | Upper | P |
|---|---|---|---|---|---|
| Size >2.5 cm | 254 | 1.795 | 1.240 | 2.600 | 0.0020* |
| Node + | 254 | 1.575 | 1.081 | 2.293 | 0.0179* |
| Grade | 254 | 1.424 | 1.069 | 1.898 | 0.0158* |
| PR (LBA | 254 | 0.614 | 0.425 | 0.886 | 0.0091* |
| >20 fmol/mg) | | | | | |

### REFERENCES

1. Weigel N, Moore N: Steroid Receptor Phosphorylation: A Key Modulator of Multiple Receptor Functions. Mol Endocrinol 21:2311-2319, 2007

2. Murphy L, Niu Y, Snell L, et al: Phospho-Serine-118 Estrogen Receptor-alpha Expression in Primary Human Breast Tumors in vivo is Associated with Better Disease Outcome in Women Treated with Tamoxifen. Clin Cancer Res 10:5902-6, 2004

3. Murphy L C, Cherlet T, Adeyinka A, et al: Phospho-Serine-118 Estrogen Receptor-alpha Detection in Human Breast Tumors in vivo. Clin Cancer Res 10:1354-1359, 2004

4. Williams C, Smith C L, Rowan B G: Identification of Four Novel Phosphorylation Sites in Estrogen Receptor α: Impact on Receptor-Dependent Gene Expression and Phosphorylation by Protein Kinase CK2, The Endocrine Society's 89th Annual Meeting. Toronto, Canada, 2007, pp P2-258

5. Al-Dhaheri M, Rowan B: Application of phosphorylation site-specific antibodies to measure nuclear receptor signaling: characterization of novel phosphoantibodies for estrogen receptor alpha. Nucl Recept Signal. 4:e007. Epub Apr. 28, 2006

6. Skliris G, Rowan B, Al-Dhaheri M, et al: Immunohistochemical validation of multiple phospho-specific epitopes for estrogen receptor α (ERα) in tissue microarrays (TMA) of ERα positive human breast carcinomas. Breast Cancer Research & Treatment, In press

7. Watson P, Snell L, Parisien M: The NCIC-Manitoba Breast Tumor Bank: a resource for applied cancer research. CMAJ 155:281-283, 1996

8. Skliris G, Leygue E, Curtis-Snell L, et al: Expression of oestrogen receptor-beta in oestrogen receptor-alpha negative human breast tumours. .Br J Cancer. 95:616-26, 2006

9. Barnes R, Parisien M, Murphy L, et al: Influence of evolution in tumor biobanking on the interpretation of ranslational research. Cancer Epidemiology, Biomarkers & Prevention (in press), 2008

10. Weigel N L, Moore N L: Kinases and protein phosphorylation as regulators of steroid hormone action. Nucl Recept Signal 5:e005, 2007

11. Cui Y, Parra I, Zhang M, et al: Elevated expression of mitogen-activated protein kinase phosphatase 3 in breast tumors: a mechanism of tamoxifen resistance. Cancer Res 66:5950-9, 2006

12. Schiff R, Massarwah S, Shou J, et al: Advanced concepts in estrogen receptor biology and breast cancer endocrine resistance: implicated role of growth factor signaling and estrogen receptor coregulators. Cancer Chemother Pharmacol 56:s10-s20, 2005

13. Norris J, Fan D, Kerner S, et al: Identification of a third autonomous activation domain within the human estrogen receptor. Mol Endocrinol 11:747-754, 1997

1. An antibody which binds to one or more phosphorylated residues of estrogen receptor α wherein said residues are selected from serine 282; serine 294; threonine 311; and serine 559.

2. A method of diagnosis said method comprising:
(a) isolating a sample from a subject having, thought to have, or at risk of developing breast cancer;
(b) exposing the sample to an antibody which binds to one or more phosphorylated residues of estrogen receptor α; and
(c) determining if the residue(s) is/are positive for phosphorylation;
wherein the residues are selected from serine 282; serine 294; threonine 311; and serine 559.

3. The method of claim 2 wherein the phosphorylation state of one or more of serine 104/106; serine 118; serine 167 of estrogen receptor α is determined using an antibody which binds to the phosphorylated form of said residues.

4. A kit comprising antibodies which bind to one or more phosphorylated residues of estrogen receptor α, wherein said residues are selected from serine 282; serine 294; threonine 311; and serine 559.

5. A kit comprising antibodies which bind to four or more phosphorylated residues of estrogen receptor α, wherein said residues are selected from serine 104/106; serine 118; serine 167; serine 282; serine 294; threonine 311; and serine 559.
